(19) United States
(12) Patent Application Publication
(10) Pub. No.: US 2010/0317849 A1
(43) Pub. Date: Dec. 16, 2010

(54) Process For Producing Pure And Stable Form Of 2-Methyl-4-(4-Methyl-1-Piperazinyl) -10H-Thieno[2,3-B] [1,5] Benzodiazepine

(76) Inventors: Dinesh Panchasara, Uttar Pradesh (IN); Poorvi Gupta, Uttar Pradesh (IN); Geetesh Kaushik, Uttar Pradesh (IN); Sushil Dubey, Uttar Pradesh (IN)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/225,080

(22) PCT Filed: Mar. 14, 2006
(86) PCT No.: PCT/IN2006/000916

§ 371 Date: Sep. 12, 2008

(51) Int. Cl. C07D 495/04

(52) U.S. Cl. 540/557

(57) ABSTRACT

Disclosed is an improved process for producing pure and thermally color stable crystalline Form I of 2-methyl-4-(4-methyl-1-piperazinyl)-10H-thieno[2,3-b][1,5]benzodiazepine and product thereof. The process comprises of reacting 2-(2-aminoanilino)-5-methylthiophene-3-carbonitrile with N-methyl piperazine in conjunction with N-methylpiperazine acid salt, to produce 2-methyl-4-(4-methyl-1-piperazinyl)-10H-thieno[2,3-b][1,5]benzodiazepine. Also disclosed is a process for obtaining the Polymorphic Form I of 2-methyl-4-(4-methyl-1-piperazinyl)-10H-thieno[2,3-b][l,5J benzodiazepine by crystallizing the crude 2-methyl-4-(4-methyl-1-piperazinyl)-10H-thieno[2,3-b][1,5]benzodiazepine in a mixture of solvents. Further the invention also provides a new polymorph of Olanzapine, dihydrate Form Ji and process for its preparation and a new hydrate Form J₂ of Olanzapine having moisture content 1-3% and process for its preparation.

### FIELD OF THE INVENTION

This invention in general relates to an improved process for producing an atypical neuroleptic or antipsychotic agent. More particularly, this invention provides an improved, concise and industrially feasible process for producing thermally color stable and pure form of 2-methyl-4-(4-methyl-1-piperazinyl)-10H-thieno[2,3-b][1,5]benzodiazepine (Olanzapine) and product thereof. Further the present invention also provides new hydrate and dihydrate forms of Olanzapine.

### BACKGROUND OF THE INVENTION

Olanzapine has antagonist activity at non-adrenergic alpha-receptors, D-1 and D-2 receptors and 5 HT-2 receptor sites. 2-Methyl-4-(4-methyl-1-piperazinyl)-10H-thieno[2,3-b][1,5]benzodiazepine (Olanzapine) is an atypical neuroleptic agent that has better reported efficacy like relaxant and iolytic or anti-emetic properties, and few side effects than conventional neuroleptic agents. It is useful in the treatment of psychotic patients suffering from schizophrenia and mild anxiety states.

U.S. Pat. No. 5,229,382 and its continuation-in-part application, now U.S. Pat. No. 6,008,216 to Chakrabarti, et al., disclose processes for Olanzapine preparation by different intermediates. One of the known procedures consists of reduction and cyclization reaction of 2-(2-nitroanilino)-5-methylthiophen-3-carbonitrile with stannous chloride (SnCl₂) in an aqueous-alcoholic solution of hydrogen chloride followed by the reaction of thus formed 4-amino-2-methyl-10H-thieno[2,3-b][1,5]benzodiazepine[3] with N-methylpiperazine in an organic solvent or solvent mixture such as anisole, toluene, dimethylformamide or dimethylsulphoxide, preferably at a temperature from 100° C. to 150° C. for about 20 hours, to which alcohol and excess water is added after the reaction is complete to get the solid product. The crude product is separated out and collected. The crude Olanzapine is then crystallized in acetonitrile and gives a crystalline form, which is designated as Form I in later patents. Form I thus formed has been reported to be metastable and changes its color on standing thus being unstable for commercial use.

Olanzapine has many polymorphic forms including hydrates and solvates as disclosed in subsequent patents and publications. U.S. Pat. No. 5,736,541 claims Form-II of Olanzapine and discloses that the product obtained according to process described in U.S. Pat. No. 5,229,382 is Olanzapine Form I. U.S. Pat. No. 5,703,232 and European Patent No. EP 0 733 634 B1 disclose solvates of Olanzapine in methanol, ethanol and isopropanol. U.S. Pat. No. 6,020,487 and European Patent No. EP 0 831 098 B1 disclose dihydrate forms of olanzapine, selected from Dihydrate B, Dihydrate D and Dihydrate E that may be used for the preparation of anhydrous olanzapine.

PCT applications WO 02/18390 and WO 03/097650 disclose the methods for preparation of polymorphic Form I of Olanzapine. PCT application WO 02/18390 relates to a method for the preparation of hydrates of Olanzapine. PCT application WO 03/097650 relates to the new mixed solvates of Olanzapine. In these applications, polymorphic Form I of Olanzapine is prepared by the conversion of hydrates and solvates of Olanzapine respectively.

In most of the prior art methods technical grade Olanzapine is separated in the form of solvate with alcohol. Depending on the solvent used for the crystallization of technical grade Olanzapine, different polymorphic forms are obtained by different prior art methods.

The other prior art patents and publications describe the different conditions and solvents for the preparation of different polymorphic forms. Polymorphism can be influenced by controlling the conditions of obtaining a compound in solid form. These polymorphic forms are mainly distinguished on the basis of IR and X-Ray diffraction data.

Prior art methods for the preparation of Olanzapine Form I have many drawbacks, such as colour change, in the process described in U.S. Pat. No. 5,229,382. Further recrystallization process as disclosed in WO 96/38151 is time consuming. The mixtures of polymorphic forms or impurities or solvates are obtained along with olanzapine in many prior art preparation processes. US Application 2003/0125322, now U.S. Pat. No. 6,906,062 reports Form I as stable form at room temperature, which is prepared from alcoholic solvent only.

The present invention provides an improved process, which overcomes the drawbacks of processes recited in prior arts. The main aim of this invention is to provide new improved forms of Olanzapine and their preparation.

### SUMMARY OF THE INVENTION

It is a principal aspect of the present invention to provide an improved, environmental friendly, industrially feasible and concise process for producing pure form of 2-methyl-4-(4-methyl-1-piperazinyl)-10H-thieno[2,3-b][1,5]benzodiazepine (Olanzapine), which is characterized by having thermal color stability.

Further aspect of the present invention is to provide new hydrates and dihydrates of olanzapine and process for producing the same.

Following embodiments further describe the different aspects of the present invention in accordance with the best mode of practice, however, disclosed invention is not restricted to the particular embodiments hereinafter described.

In accordance with one preferred embodiment, the present invention provides an improved environment friendly process for producing a pure form of Olanzapine, by reacting 2-(2-aminoanilino)-5-methylthiophene-3-carbonitrile with N-methyl piperazine in conjunction with N-methyl piperazine acid salt in a one step reaction without employing any solvent, at a temperature of 90-138° C., preferably 110-125° C., wherein the ratio of 2-(2-aminoanilino)-5-methylthiophene-3-carbonitrile and N-methyl piperazine is more than 1:4 weight by volume.

In accordance with another embodiment, the present invention provides for an improved, industrially feasible and concise process for producing pure form of Olanzapine, by reacting 2-(2-aminoanilino)-5-methylthiophene-3-carbonitrile with N-methyl piperazine in conjunction with N-methyl piperazine acid salt in presence of a solvent. The solvent can be selected from toluene, dimethylsulfoxide, n-butanol, methyl ethyl ketone, dimethyl formamide, or a mixture thereof.

In accordance with still another preferred embodiment, the present invention provides an improved process for producing Olanzapine by condensing 4-amino-2-methyl-10H-thieno[2,3-b][1,5]benzodiazepine or its hydrochloride salt with N-methyl piperazine at 90 to 138° C. without employing any solvent, wherein the ratio of 4-amino-2-methyl-10H-thieno[2,3-b][1,5]benzodiazepine and N-methyl piperazine is more than 1:4 weight by volume. It is surprisingly found that the absence of solvents lead to reduction in reaction time to 2-3 hours, as compared to 20 hours reported in prior art.

In accordance with another preferred embodiment the N-methylpiperazine acid salt is prepared in situ in the reaction mass or prepared separately and added to the reaction mass.

In accordance with still another embodiment, the present invention provides an improved process for obtaining a polymorphic Form I of Olanzapine by producing the crude Olanzapine according to the above-mentioned processes and crystallizing the same in a mixture of two or more solvents, wherein the solvents are selected from a group comprising acetonitrile, dichloromethane, diisopropylether, cyclohexane, hexane, t-butyl methyl ether and propionitrile.

In accordance with still another embodiment, the present invention provides an improved process for obtaining a polymorphic Form I of Olanzapine by producing the crude Olanzapine according to the above-mentioned process and crystallizing the same in a mixture of dichloromethane and diisopropylether or cyclohexane.

In accordance with still another embodiment, the present invention provides an improved process for obtaining a polymorphic Form I of Olanzapine by producing the crude Olanzapine according to the above-mentioned process and crystallizing the same in a mixture of dichloromethane and acetonitrile.

In accordance with another embodiment, the form I is produced from crude Olanzapine, which is free from other polymorphic forms, prepared according to the above-mentioned processes, crystallizing the same in a mixture of two or more solvents as described above, further characterized by having stable colour upon storage at temperature of 40° C. and 75% relative humidity.

In accordance with still another embodiment of the present invention, there is provided the new dihydrate Form J₁ of Olanzapine and process for preparing the same.

In accordance with yet another embodiment of the present invention, there is provided a process for preparing said stable Form I of Olanzapine using dihydrate Form J₁ of Olanzapine.

In accordance with still another embodiment of the present invention, there is provided a hydrate Olanzapine Form J₂ and its process for preparation by exposing Form I in controlled humidity conditions.

### BRIEF DESCRIPTION OF DRAWINGS

Further objects of the present invention together with additional features contributing thereto and advantages accruing there from will be apparent from the description of preferred embodiments of the present invention which are shown in the accompanying drawing figures.

FIG. 1 is an X-ray powder diffraction pattern corresponding to Form I of Olanzapine.

FIG. 2 is an X-ray powder diffraction pattern corresponding to hydrate Olanzapine Form J₂.

FIG. 3 is thermo gravimetric analysis thermogram of hydrate Olanzapine Form J₂.

FIG. 4 is differential scanning calorimetry thermogram of hydrate Olanzapine Form J₂.

FIG. 5 is an x-ray powder diffraction pattern corresponding to dihydrate Form J₁ of Olanzapine.

FIG. 6 is differential scanning calorimetry thermogram of dihydrate Olanzapine Form J₁.

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included drawings.

According to the preferred embodiments of the invention, there is provided a pure and thermally color stable form of 2-methyl-4-(4-methyl-1-piperazinyl)-10H-thieno[2,3-b]1,5-benzodiazeine[1], commonly known as Olanzapine by an improved process.

The process disclosed in the present invention is performed without employing additional solvent, which leads to a less cumbersome workup, which is more suitable for large-scale manufacturing of pure Olanzapine. Further, the absence of solvents saves the cost in terms of raw material, since recovery of the solvent is not needed and therefore utility costs are also saved. The process is environment friendly, as the vapors of solvents are not spread in the atmosphere.

N-Methylpiperazine acid salt can be prepared in situ or can be prepared separately and then added into the reaction mixture. N-Methylpiperazine acid salt is prepared by the reaction of N-methylpiperazine with an acid in usual fashion. The acid used to prepare N-methylpiperazine acid salt can be chosen from organic or inorganic acids. The preferred organic acids can be formic acid, substituted or unsubstituted acetic acid e.g. acetic acid or trifluoroacetic acid, alkyl, aryl or aralkyl sulphonic acid e.g. methane sulphonic acid, p-toluene sulphonic acid, substituted or unsubstituted benzoic acid, etc. The preferred inorganic acids are phosphoric acid, hydro halide acid e.g. hydrochloric acid, sulfuric acid, perchloric acid and lewis acids such as aluminium chloride.

The solvent does not play any role in the reaction and reaction proceeds well in absence of any solvent, but alternatively the reaction can be performed in presence of a solvent. The solvent can be selected from toluene, dimethylsulfoxide, n-butanol, methyl ethyl ketone, dimethyl formamide, or a mixture thereof.

After the completion of reaction, water miscible or water immiscible solvent is added followed by the addition of water. In case of water miscible solvents addition of water forms crude olanzapine directly, whereas in case of water immiscible solvent, solvent is removed to obtain crude Olanzapine. This crude Olanzapine is dried at ambient temperature and crystallized in different solvents or solvent systems to obtain different crystallized forms of Olanzapine, as desired. The solvent used for the work up can be selected from chlorinated solvent, amidic solvent, ketonic solvent, ethereal solvent, ester solvent, etc. Few examples, but not limited are dimethylformamide, tetrahydrofuran, dioxane, acetone, acetonitrile, ethyl acetate or dichloromethane.

According to the present invention, the stable Form I of Olanzapine is prepared by dissolving the crude Olanzapine in a mixture of two or more solvents, concentrating the reaction mixture, distilling the reaction mixture and removing the solvents up to 30-50% by volume, cooling the resulting reaction mixture to a temperature in the range of 0-20° C., preferably 0-5° C., crystallizing the solid and, filtering the material and drying preferably at a temperature in the range of 45-50° C. under vacuum to obtain the crystallized Form I of Olanzapine.

The crystallized Form I obtained is highly pure (>99.6%) and colour stable upon storage at high temperature at 40° C.

The solvents used in the above-mentioned process are selected from a group comprising acetonitrile, dichloromethane, diisopropylether, cyclohexane, hexane, t-butyl methyl ether and propionitrile. The ratio of the two solvents in solvent mixture is preferably 1:1.

Crystalline stable Olanzapine Form I is characterized by their X-ray powder diffraction pattern. Thus the X-ray diffraction patterns of Olanzapine Forms are measured on a PANalytical X′ Pert Pro diffractometer with Cu radiation and expressed in terms of 2θ, d-spacing and relative intensities.

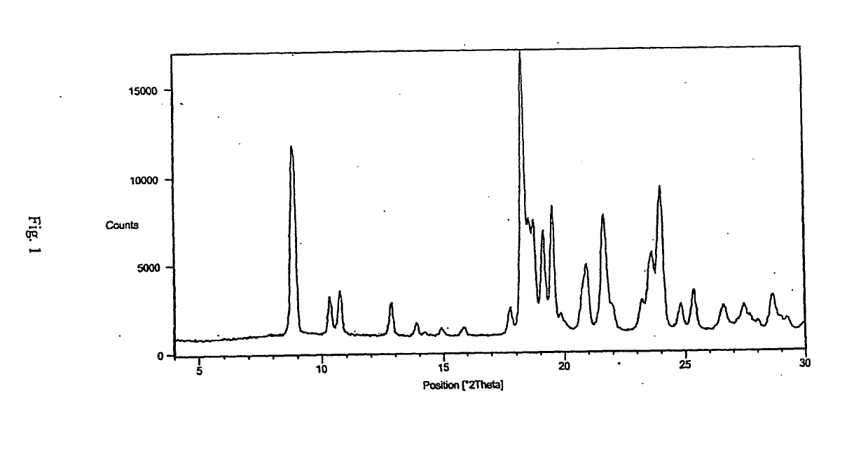

The X-Ray powder diffraction of Form I of Olanzapine is given in FIG. 1 and is characterized by peaks at 8.9, 10.3, 10.7, 12.8, 14.2, 17.8, 18.3, 18.8, 19.2, 19.5, 20.7, 21.0, 21.6, 23.2, 24.1, 25.4, 28.6±0.2 degrees 2 theta. The Form I is having stable colour at all temperatures including the high temperature (40° C.) and high moisture condition or under stress conditions. The stable Form I is also produced when Olanzapine is crystallized directly from the reaction mixture using the same conditions as described above.

The crude Olanzapine is prepared either by any prior art method or by condensing 2-(2 aminoanilino)-5-methylthiophene-3-carbonitrile with N-methyl acid piperazine in conjunction with N-methylpiperazine salt in absence of any solvent at a temperature of 90-140° C.

Table 1 shows the stability of Olanzapine Form I at stressed conditions upto 6 months (40° C.) wherein the HPLC purity, total impurity and colour is given (UV absorbance at 430 nm). The data clearly shows that olanzapine is having high stability for three months under stressed conditions.

TABLE 1

| | | Impurity Profile | | Absorbance |
|---|---|---|---|---|
| | | Total | Chroma- | at |
| | | Imp. | tographic | 430 nm |
| SR. No. | Conditions | % age | purity % | Log I/Io |
| 1 | Initial | 0.03 | 99.97 | 0.4 |
| 2 | 30 DAYS/40° C./75% RH | 0.06 | 99.94 | 0.402 |
| 3 | 60 DAYS/40° C./75% RH | 0.04 | 99.96 | 0.407 |
| 4 | 120 DAYS/40° C./75% RH | 0.05 | 99.95 | 0.4034 |
| 5 | 180 DAYS/40° C./75% RH | 0.07 | 99.93 | 0.4026 |

The colour of Olanzapine is determined by following method:

Olanzapine 50 mg was dissolved in 50 ml 0.1 N HCl in methanol, which was diluted to prepare 100 μgm/ml, 200 μgm/ml, 500 μgm/ml of Olanzapine solution. Absorbance was observed at 430 nm using 0.1 N HCl in methanol as blank by using UV visible spectrophotometer Perkin Elmer Lambda-35. Calibration curve was plotted between absorbance and concentration.

% of Colour=Amount of colour from calibration curve×100/Weight of sample taken.

The Table-1 shows that the Form I thus produced is highly stable at stress conditions. Thus it is highly suitable for use in formulation.

The new polymorph of Olanzapine dihydrate Form J₁ according to the present invention is prepared by a process comprising dissolving the Olanzapine in the mixture of water and ethyl acetate and stirring the mixture at room temperature, heating the reaction mixture gradually preferably at a temperature in the range of 75-80° C. to obtain a clear solution, stirring the solution preferably at a temperature in the range of 75-80° C. for a period in the range of 0.5-2.0 hours preferably for a period in the range of 15-30 minutes, cooling the solution to a temperature in the range of 0-30° C., preferably 0-5° C. for a period in the range of 0.5 to 2 hours and filtering the material and suck drying to obtain a new polymorphic Form J₁.

Form J₁ of Olanzapine is characterized by X-ray powder diffraction pattern and Differential Scanning Calorimetry. DSC thermogram is recorded on DSCQ100 equilibrated at 25° C. to 350° C. at 10° C./minute with nitrogen flow rate of 60 ml/minute.

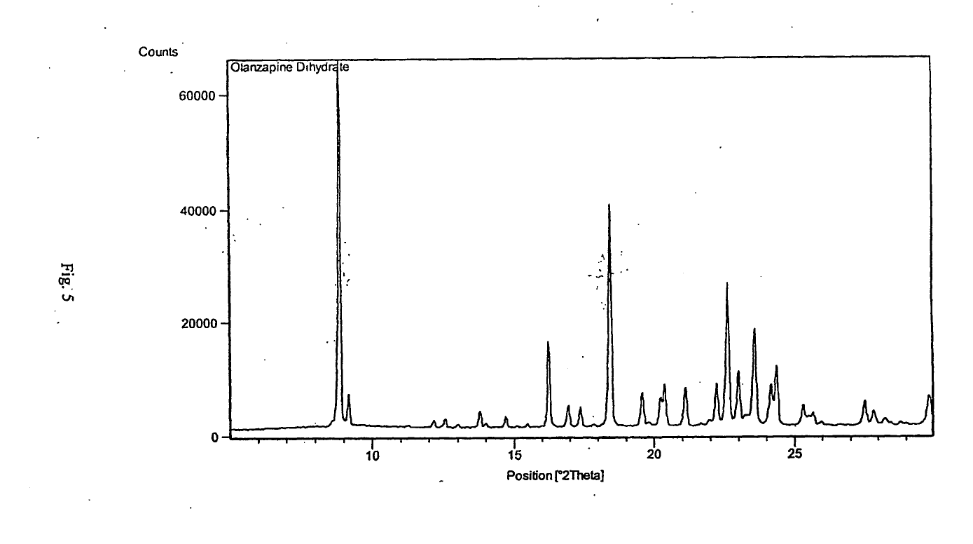

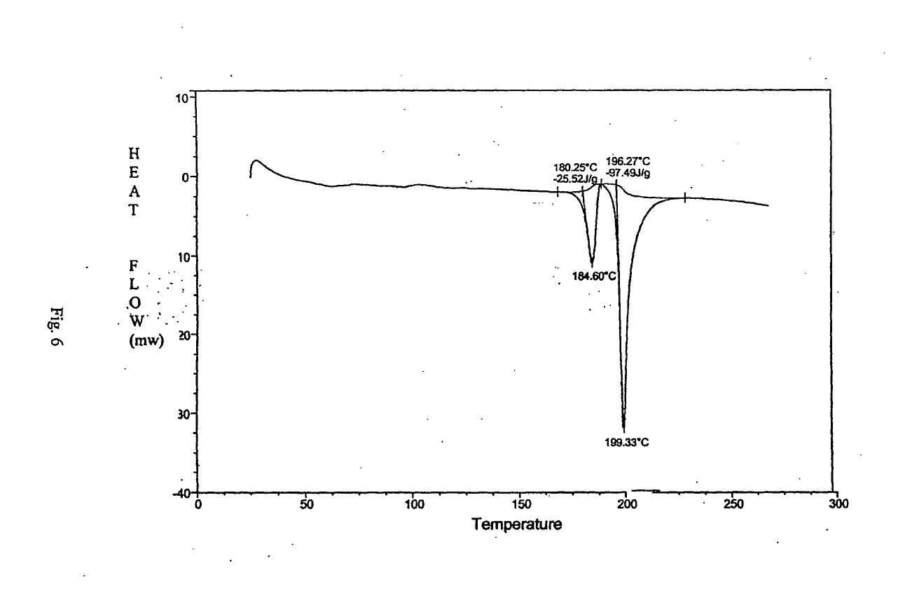

The reference X-Ray powder diffraction and DSC thermogram analyses are attached herein as FIGS. 5 and 6. The XRD has characteristic peaks at about 8.9, 18.4±0.2 degrees two theta and at about 16.2, 20.3, 21.1, 22.2, 22.6, 23.0, 23.5, 24.1, 24.3±0.2 degrees two theta. The new polymorphic Form J₁ contains the moisture content of about 10.5% according to Karl Fisher analysis and ethyl acetate approximately 0.7%.

Further the obtained new polymorphic Form J₁ is used for preparing said Form I of Olanzapine, the process comprising, contacting a new polymorphic Form J₁ with cyclohexane/dichloromethane at room temperature, refluxing the reaction mixture, removing the water from material by azeotropic distillation, filtering the material and separating the solid mass, dissolving this solid mass in dichloromethane, cooling and stirring the resulting mixture to a temperature in the range of 0-5° C. for a period in the range of 0.5-2 hours, filtering the mixture and sucking dry/drying the solid mass under vacuum at a temperature in the range of 30-35° C. to obtain stable Form I, which is further characterized in that having stable colour upon storage at high temperature at 40° C. and 75%.

The disclosed new hydrate Olanzapine Form J₂ according to the present invention is prepared by a process comprising exposing the Olanzapine Form I in an open flask, further exposing the material to humidity conditions for more than 24 hours, controlling the humidity conditions, absorbing the water by material, which gives a new hydrate Form J₂ of Olanzapine, drying of hydrate Olanzapine Form J₂ at 45-55° C. under vacuum to get Olanzapine Form I which is confirmed by XRD, DSC and TGA.

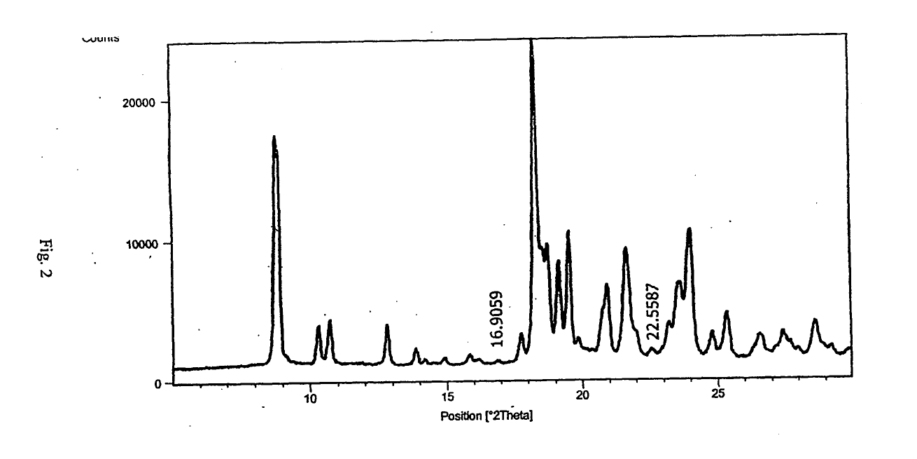

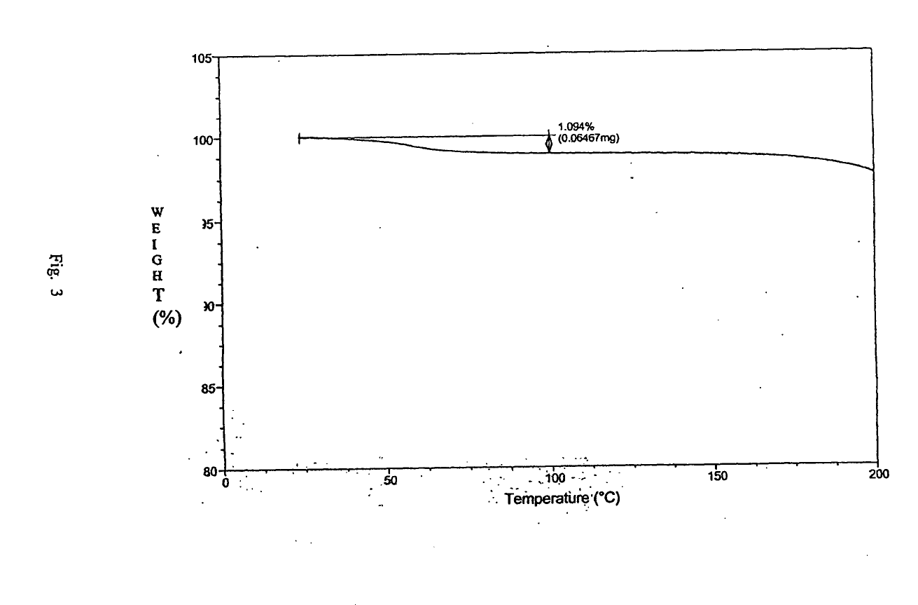

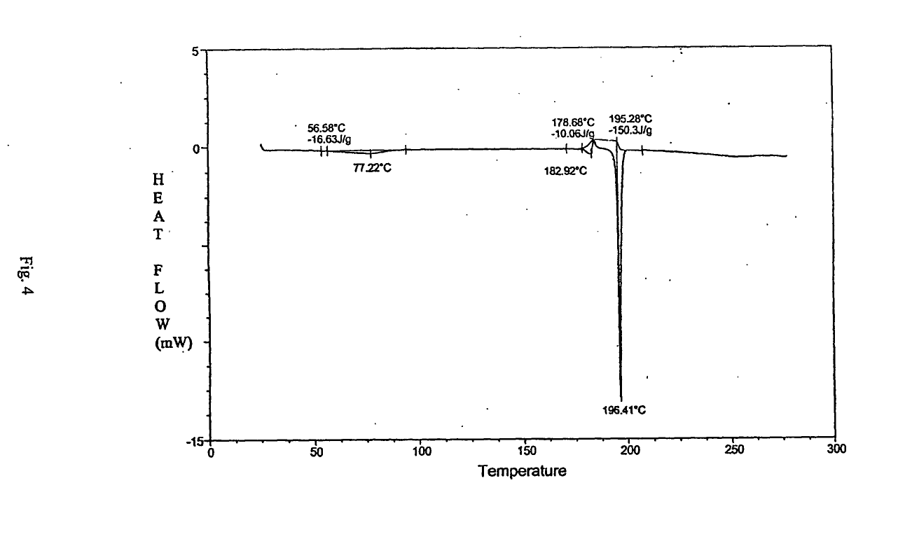

The hydrate Form J₂ of Olanzapine is characterized by X-ray powder diffraction pattern (PXRD), Thermo Gravimetric Analysis (TGA) and Differential Scanning Calorimetry (DSC). The reference X-Ray powder diffractogram, Thermo gravimetric analyses and Differential Scanning Calorimetry thermogram are attached herein FIGS. 2, 3 and 4.

The XRD has characteristic peaks at about 8.79, 8.9, 10.7, 12.8, 18.3, 18.5, 18.7, 19.1, 19.5, 20.7, 20.9, 21.6, 21.7, 23.5, 23.6, 24.0, 25.3±0.2 degrees two theta. The other characteristic peaks are 16.1, 22.5±0.2 degrees two theta. TGA thermogram is recorded on TGA Q 50 with a ramp of 10° C./min to 200° C. with nitrogen flow rate at 60 ml/minute. The hydration level of hydrate Olanzapine Form J₂ is indicated by a weight loss of 1.108% at about 100° C. by TGA. DSC thermogram is recorded on DSCQ100 equilibrated at 25° C. to 280° C. at ramp 10° C./minute with nitrogen flow rate at 60 ml/minute. A typical DSC scan of hydrate Olanzapine Form J₂ shows an endothermic peak at about 60°-80° due mainly to water, and a subsequent endotherm at about at 196.4° C. with a small endotherm peak also at 182.9° C. The moisture content of this hydrate form is about 1.2-3.0%, measured by Karl Fisher technique.

For the purpose of promoting a further understanding of the present invention and its preferred features and embodiments, the following examples are being provided. It will be understood, however, that these examples are illustrative, and not limiting in nature.

### Example 1

### Preparation of 2-amino-5-methylthiophene-3-carbonitrile

A mixture of sulphur (46 g), propionaldehyde (100 g) and dimethylformamide (200 ml) were taken under nitrogen. Triethylamine (113.0 ml) was added to the reaction mixture and cooled to 0-5° C. A solution of malonitrile (95 g) in dimethylformamide (200 ml) was further added to the reaction mixture. After addition, the reaction mixture was stirred for 45 minutes. The reaction mixture was then poured into ice water (2400 ml). The solid thus obtained was isolated by filtration, washed with chilled water and the product was air-dried to obtain the title compound (140 g).

### Example 2

### Preparation of 2-(2-nitroanilino)-5-methylthiophene-3-carbonitrile

Potassium hydroxide (101 g) in acetonitrile (150 ml) was taken under nitrogen and cooled to 0-5° C. A solution of 2-amino-5-methylthiophene-3-carbonitrile (100 g) and ortho-fluoronitrobenzene (122 g) in acetonitrile (550 ml) was added. The reaction mixture was then stirred for 3 hours and chilled water was added into the reaction mixture. The solid was separated out. The solid thus obtained was filtered off and air-dried. The solid was crystallized from water-methanol mixture and the crystallized solid was dried under vacuum at 40-45° C. to obtain the title compound (140 g).

### Example 3

### Preparation of 2-(2-aminoanilino)-5-methylthiophene-3-carbonitrile

2-(2-Nitroanilino)-5-methylthiophene-3-carbonitrile (100 g) was taken in ethyl acetate (1000 ml). The reaction mixture was then hydrogenated by 5% Pd/C (15 g) at 50-55° C. and at 10-12 Kg pressure. The reaction mixture was filtered, and ethyl acetate was distilled off to get the title compound.

### Example 4

### Preparation of 2-methyl-4-(4-methyl-1-piperazinyl)-10H-thieno-[2,3-b][1,5]benzodiazepine (Olanzapine)

2-(2-Aminoanilino)-5-methylthiophene-3-carbonitrile (2 g) was suspended in N-methyl-piperazine (12 ml) and 4.5 g of N-methyl piperazine hydrochloride was added. The reaction mixture was heated at 120° C. until the reaction was complete. The reaction mass was cooled to 70-75° C. and acetone and activated charcoal were added. The reaction mixture was stirred for 30 minutes and filtered. The water was added at 45-50″C, the mixture was cooled up to room temperature and the precipitated solid was filtered off and washed with acetone-water mixture.

### Example 5

### Preparation of 2-methyl-4-(4-methyl-1-piperazinyl)-10H-thieno-[2,3-b][1,5]benzodiazepine

2-(2-Aminoanilino)-5-methylthiophene-3-carbonitrile (10 g) was taken in N-methyl-piperazine (60 ml) and N-methyl-piperazine hydrochloride (24 gm). The solution was heated at 120° C. until completion of reaction. The reaction mass was cooled and dichloromethane (100 ml) and water were added. The mixture was cooled to room temperature and dichloromethane layer was separated. Dichloromethane (50 ml) was evaporated and cyclohexane was added to get a clear solution. On cooling the solution, solid was separated out, which was filtered and dried under vacuum to get Olanzapine Form I (Purity>99.6%)

### Example 6

### Preparation of Polymorphic Form I

The crude Olanzapine was dissolved in a mixture of dichloromethane (5 times) and diisopropylether or cyclohexane (5 times). The solution was cooled to give solid. Crystallized solid obtained was filtered and dried under vacuum at 45-50° C. to give Form I (Purity>99.6%)

### Example 7

### Preparation of hydrate Form J₂ of 2-methyl-4-(4-methyl-1-piperazinyl)-10H-thieno-[2,3-b][1,5]benzodiazepine

Form I (5 g) was taken in the open flask and exposed to humidity for more then 24 hours. Hydrate Olanzapine Form J₂ was collected with moisture content 1.3%. PXRD, TGA and DSC taken are given in FIGS. 2, 3 and 4.

### Example 8

### Preparation of Olanzapine Form I from Hydrate Form J₂

Hydrate Form J₂ (5 g) was dried in oven under vacuum at 45-50° C. to give Form I. XRD, DSC and TGA matched with Form I.

### Example 9

### Preparation of dihydrate Olanzapine Form J₁ of 2-methyl-4-(4-methyl-1-piperazinyl)-10H-thieno-[2,3-b][1,5]benzodiazepine

Olanzapine (10 g) was taken in 100 ml ethyl acetate (m/c-3.8%) and stirred at room temperature. The contents of the flask were gradually heated to 75-80° C. to obtain a clear solution and then stirred for 15 minutes at 75-80° C. The reaction mixture was cooled to 0-5° C. The reaction mixture was stirred for one hour at 0-4° C. The solid product was filtered and suck dried to give a new polymorphic Form J₁ having moisture content 10.5%. The percentage of ethyl acetate in the Form J₁ is approximately 0.7%.

### Example 10

### Preparation of Stable Form I from Form J₁

The new Form J₁ (10 g) was taken in 100 ml cyclohexane at room temp. The contents of the flask were gradually heated to reflux temperature. Water was removed from material by azeotropic distillation. The obtained residue mass was dissolved in dichloromethane (50 ml) at 45-50° C. The reaction mixture was cooled to 0-5° C. The reaction mixture was stirred for one hour at same temperature. The solid product was filtered and suck dried to give Form I.

### Example 11

### Preparation of Stable Form I from Form J₁

The new Form J₁ (10 g) was taken in dichloromethane (150 ml) in 250 ml round bottom flask. Water from material was removed by azeotropic distillation to give a residue. Dichloromethane (50 ml.) was added. The flask was allowed to cool to 0-5° C. and stirred the residue for one hour at same temperature. The solid was separated. The solid product was filtered and dried under vacuum at 30° C. to give Form I.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

1. A crystalline Form I of 2-methyl-4-(4-methyl-1-piperazinyl)-10H-thieno[2,3-b][1,5]benzodiazepine having colour stability upon storage at high temperature.

2. The crystalline Form I of 2-methyl-4-(4-methyl-1-piperazinyl)-10H-thieno[2,3-b][1,5]benzodiazepine according to claim 1, wherein said Form I has colour stability upon storage under stressed conditions or at 40° C.

3. The crystalline Form I of 2-methyl-4-(4-methyl-1-piperazinyl)-10H-thieno[2,3-b][1,5]benzodiazepine according to claim 1, wherein said Form I has colour stability upon storage under stressed conditions or at high moisture conditions.

4. The crystalline Form I of 2-methyl-4-(4-methyl-1-piperazinyl)-10H-thieno[2,3-b][1,5]benzodiazepine according to claim 1, wherein said Form I is produced in accordance with a process comprising crystallizing crude Olanzapine employing dichloromethane-cyclohexane or a diisopropyl ether mixture.

5. The crystalline Form I of 2-methyl-4-(4-methyl-1-piperazinyl)-10H-thieno[2,3-b][1,5]benzodiazepine according to claim 1, wherein said Form I is produced in accordance with a process comprising preparing crude Olanzapine by reacting 2-(2-aminoanilino)-5-methylthiophene-3-carbonitrile with N-methyl piperazine in conjunction with N-methyl piperazine acid salt and crystallizing the obtained resultant by dissolving in dichloromethane-cyclohexane or a diisopropyl ether mixture.

6. The crystalline Form I of 2-methyl-4-(4-methyl-1-piperazinyl)-10H-thieno[2,3-b][1,5]benzodiazepine according to claim 1, wherein said Form I is characterized by a powder X-ray diffraction pattern with characteristic peaks at 8.9, 10.3, 10.7, 12.8, 14.2, 17.8, 18.3, 18.8, 19.2, 19.5, 20.7, 21.0, 21.6, 23.2, 24.1, 25.4, 28.6±0.2 degrees 2 theta.

7. A hydrate Form J₂ of 2-methyl-4-(4-methyl-1-piperazinyl)-10H-thieno[2,3-b][1,5]benzodiazepine having a moisture content of 1-3%.

8. The hydrate Form J₂ of 2-methyl-4-(4-methyl-1-piperazinyl)-10H-thieno[2,3-b][1,5]benzodiazepine according to claim 7, wherein said Form J₂ is characterized by powder a X-ray diffraction pattern with characteristic peaks at 16.1 and 21.5 d±0.2 degrees two theta value.

9. The hydrate Form J₂ of 2-methyl-4-(4-methyl-1-piperazinyl)-10H-thieno[2,3-b][1,5]benzodiazepine according to claim 8, said hydrate form J₂ having a powder X-ray diffraction pattern essentially as depicted in FIG. 2.

10. The hydrate Form J₂ of 2-methyl-4-(4-methyl-1-piperazinyl)-10H-thieno[2,3-b][1,5]benzodiazepine according to claim 7, wherein said Form J₂ is prepared by a process comprising:
exposing the 2-methyl-4-(4-methyl-1-piperazinyl)-10H-thieno[2,3-b][1,5]benzodiazepine Form I in an open flask;
exposing the material to humid conditions for more than 24 hours; and
collecting the resultant hydrate Form J₂.

11. A process for preparing crystalline Form I of 2-methyl-4-(4-methyl-1-piperazinyl)-10H-thieno[2,3-b][1,5]benzodiazepine comprising drying hydrate Form J₂ produced according to claim 10 under vacuum at a temperature of 45-55° C.

12. A dihydrate Form J₁ of 2-methyl-4-(4-methyl-1-piperazinyl)-10H-thieno[2,3-b][1,5]benzodiazepine having a moisture content of 10-11%.

13. The dihydrate Form J₁ of 2-methyl-4-(4-methyl-1-piperazinyl)-10H-thieno[2,3-b][1,5]benzodiazepine according to claim 12, wherein said Form J₁ is characterized by powder a X-ray diffraction pattern with characteristic peaks at about 8.9 and 18.4±0.2 degrees two theta.

14. The dihydrate Form J₁ of 2-methyl-4-(4-methyl-1-piperazinyl)-10H-thieno[2,3-b][1,5]benzodiazepine according to claim 12, wherein said Form J₁ is characterized by a powder X-ray diffraction pattern with characteristic peaks at about 16.2, 20.3, 21.1, 22.2, 22.6, 23.0, 23.5, 24.1, 24.3±0.2 degrees two theta.

15. The dihydrate Form J₁ of 2-methyl-4-(4-methyl-1-piperazinyl)-10H-thieno[2,3-b][1,5]benzodiazepine according to claim 14, said dihydrate Form J₁ having a powder X-ray diffraction pattern essentially as depicted in FIG. 5.

16. A process for preparing dihydrate Form J₁ of 2-methyl-4-(4-methyl-1-piperazinyl)-10H-thieno[2,3-b][1,5]benzodiazepine according to claim 12 comprising:
dissolving Olanzapine in ethyl acetate by heating;
adding water and cooling the solution; and
filtering Olanzapine dihydrate Form J₁ as solid.

17. A process for preparing crystalline Form I of 2-methyl-4-(4-methyl-1-piperazinyl)-10H-thieno[2,3-b][1,5]benzodiazepine comprising:
contacting the dihydrate Form J₁ of 2-methyl-4-(4-methyl-1-piperazinyl)-10H-thieno[2,3-b][1,5]benzodiazepine according to claim 12 in water-immiscible solvent;
removing water azeotropically;
and
collecting the solid 2-methyl-4-(4-methyl-1-piperazinyl)-10H-thieno[2,3-b][1,5]benzodiazepine as Form I.

18. The process according to claim 17, wherein the water-immiscible solvent is a chlorinated solvent or a hydrocarbons.

19. The process according to claim 18, wherein the chlorinated solvent is selected from dichloromethane and chloroform.

20. The process according to claim 18, wherein the hydrocarbon is cyclohexane.

21. The process of claim 17, wherein following the step of removing water azeotropically, the process further comprises the step of removing part of the solvent and cooling the solvent.
